(19) United States
(12) Patent Application Publication
(10) Pub. No.: US 2022/0185905 A1
(43) Pub. Date: Jun. 16, 2022

(54) MULTISPECIFIC AGENTS FOR TREATMENT OF CANCER

(71) Applicant: Forty Seven, Inc., Foster City, CA (US)

(72) Inventors: Mark McCamish, Ventura, CA (US); Jens-Peter Volkmer, Menlo Park, CA (US)

(21) Appl. No.: 17/442,593

(22) PCT Filed: Mar. 25, 2020
(86) PCT No.: PCT/US20/24707

§ 371 Date: Sep. 23, 2021

Related U.S. Application Data
(60) Provisional application No. 62/824,213, filed on Mar. 26, 2019.

(51) Int. Cl. C07K 16/28; C07K 14/705; A61P 35/00

(52) U.S. Cl. CPC C07K 16/2896; C07K 16/2803; C07K 14/70503; A61K 39/00; A61P 35/00; C07K 2317/31; C07K 16/2887

(57) ABSTRACT

The invention provides multispecific agents having one arm binding CD47, and a second arm binding to CD24. The invention also provides multispecific agents having one arm binding to SIRPα and a second arm binding to siglec-10.

### CROSS-REFERENCE TO RELATED APPLICATION

This application claims priority from U.S. 62/824,213 filed Mar. 26, 2019, incorporated by reference in its entirety for all purposes.

### SEQUENCE LISTING

This application discloses sequences contained in a txt sequence listing named 20-03-25 544571SL, of 7 kbytes, created Mar. 25, 2020, which is incorporated by reference.

### BACKGROUND

CD47 is a broadly expressed transmembrane glycoprotein with a single Ig-like domain and five transmembrane regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells. CD47 mediates a variety of biological processes, including leukocyte adhesion and migration, T-cell activation, apoptosis and phagocytosis.

Binding of SIRPα on macrophages to CD47 expressed on the host target cell generates an inhibitory signal mediated by SHP-1 that negatively regulates phagocytosis. SIRPα acts to negatively control innate immune effector function against host cells.

CD47 is also constitutively upregulated on a number of cancers, including hematopoietic cancers and solid tumors. Overexpression of CD47 increases pathogenicity of cancers by allowing the cancer cells to evade phagocytosis. Although CD47 represents a target for treatment of cancer, expression of CD47 on normal cells, particularly red blood cells, can result in off-target effects.

Like CD47, CD24 is expressed in many normal tissues, and at elevated levels in many cancers. One of the counterreceptors for CD24 is known as siglec G (mouse) or siglec-10 (human). Siglec G/10 is expressed primarily on B cells, cells of monocyte lineage and esoinophils. The CD24-siglec G/10 pathway discriminates between pathogen-associated molecular patterns (PAMPs) from Danger Associated Molecular Patterns (DAMPs) by selective repression of the host response to DAMPs. DAMPs but not PAMPs bring CDS24 Siglec G/10 into the proximity of TLR/NLR, thus allowing siglec G/10-associated phosphatases, such as SHP1 to repress the DAMP-initiated TLR/NLR signaling. Soluble forms of CD24 are in development for treatment of autoimmune diseases.

Binding of CD24 to siglec G/10 in cis or trans results in signaling through an ITIM motif of siglec G/10 causing SHP-1 inhibition of inflammatory pathways. Thus, the interaction of CD24 with siglec G/10 results in parallel anti-inflammatory effects to interaction of CD47 with SIRPα.

### SUMMARY OF THE CLAIMED INVENTION

The invention provides a multispecific agent comprising a first binding arm that specifically binds CD47 and a second binding arm that specifically binds to CD24. Optionally the first binding arm antagonizes CD47 binding to SIRPα and the second binding arm antagonizes CD24 binding to siglec-10. Optionally, the first binding arm is an antibody VH-VL pair or a SIRPα extracellular domain, and second binding arm is an antibody VH-VL pair or a siglec-10 extracellular domain. Optionally, the multispecific agent has a single first binding arm and a single second binding arm. Optionally the multispecific agent has two copies of a first binding arm and two copies of a second binding arm. Optionally, the multispecific agent further comprises a third binding arm specifically binding to a cancer antigen. Optionally, the cancer antigen is CD20. Optionally, the first and second binding arms have affinities for CD47 and CD24 within a factor of four for one another. Optionally, the second binding arm has a higher affinity for CD24 by at least a factor of five than the first binding arm has for CD47. Optionally, the multispecific agent further comprises an Fc domain. Optionally, the Fc domain is of human IgG4 isotype. Optionally, the Fc domain of human IgG1 or IgG4 isotype. Optionally, the multispecific agent is of human IgG1 isotype mutated to reduce effector functions.

The invention further provides a method of treating a patient having a cancer, comprising administering a multispecific agent to the patient. Optionally, the cancer expresses CD24 and CD47. Optionally, the multispecific agent further comprises a third binding arm specifically binding to a cancer antigen, wherein the cancer expresses the cancer specific antigen. Optionally, the cancer is adenocarcinoma. Optionally, the cancer is a lymphoma. Optionally, the method further comprises detecting expression of CD24 and CD47 on cells of the cancer.

The invention further provides a multispecific agent comprising a first binding arm that specifically binds to SIRPα and a second binding arm that specifically binds to siglec-10. Optionally, the first binding arm antagonizes CD47 binding to SIRPα and the second binding arm antagonizes CD24 binding to siglec-10. Optionally, the first binding arm is an antibody VH-VL pair or a SIRPα extracellular domain, and second binding arm is an antibody VH-VL pair or a siglec-10 binding domain. Optionally, the multispecific agent has a single first binding arm and a single second binding arm. Optionally, the multispecific agent has two copies of a first binding arm and two copies of a second binding arm. Optionally, the first and second binding arms have affinities for SIRPα and siglec-10 within a factor of four for one another. Optionally, the second binding arm has at least five fold higher affinity for siglec-10 than the first binding arm has for SIRPα. Optionally, the multispecific agent further comprises an Fc domain. Optionally, the Fc domain is of human IgG4 isotype. Optionally, the Fc domain of human IgG1 or IgG4 isotype. Optionally, the Fc domain is of human IgG1 isotype mutated to reduce effector functions.

The invention further provides a method of treating a patient having a cancer, comprising administering a multispecific agent comprising a first binding arm specifically binding to SIRPα and a second binding arm specifically binding to siglec-10 to the patient. Optionally, the multispecific agent further comprises a third binding arm specifically binding to a cancer antigen, wherein the cancer expresses the cancer specific antigen.

### BRIEF DESCRIPTION OF THE DRAWINGS

FIG. 1 shows expression of CD24 in cancers and tissue matched normal tissue.

FIG. 2 shows expression of CD24 in lymphomas.

FIG. 3 shows expression of siglec-10 in cancers and tissue matched normal tissue.

FIG. 4 shows macrophage phagocytosis of colorectal adenocarcinoma by various antibodies.

FIG. 5 shows macrophage-mediated phagocytosis of ovarian adenocarcinoma by various antibodies.

### DEFINITIONS

Multispecific agents of the invention are typically provided in isolated form. This means that a multispecific agent is typically at least 50% w/w pure of interfering proteins and other contaminants arising from its production or purification but does not exclude the possibility that the multispecific agent is combined with an excess of pharmaceutical acceptable carrier(s) or other vehicle intended to facilitate its use. Sometimes multispecific agents are at least 60, 70, 80, 90, 95 or 99% w/w pure of interfering proteins and contaminants from production or purification. Often a multispecific agent is the predominant macromolecular species remaining after its purification.

Specific binding of a multispecific agent to its target antigens means an affinity of at least 10⁶, 10⁷, 10⁸, 10⁹, or 10¹⁰ M⁻¹. Affinities can be different for the different targets. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a multispecific agent with two different binding sites binds only against targets for these two binding sites.

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the multispecific agents of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. In IgA, the heavy constant region is divided into CH1, CH2 and CH3. IgM includes constant region domains Cμ1, Cμ2, Cμ3, Cμ4 a tailpiece of 20 amino acids. The CH1 region binds to the light chain constant region by disulfide and noncovalent bonding. The hinge region provides flexibility between the binding and effector regions of an antibody and also provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, α, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, in bispecific antibodies, these binding sites can be the same or different depending on the format (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chain variable regions or between different light chain variable regions are assigned the same number. Although Kabat numbering can be used for antibody constant regions, the EU index (also called EU numbering) is more commonly used, as is the case in this application.

The term “epitope” refers to a site on an antigen to which an arm of a multispecific agent binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “antigen” or “target antigen” indicates a target molecule bound by one binding site of a multispecific agent. An antigen may be a protein of any length (natural, synthetic or recombinantly expressed), a nucleic acid or carbohydrate among other molecules. Antigens include receptors, ligands, counter receptors, and coat proteins.

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2 times, 5 times, 10 times, 20 times or 100 times) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “subject” includes human and other mammalian subjects that receive either prophylactic or therapeutic treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Nonconservative substitutions constitute exchanging a member of one of these classes for a member of another.

Percentage sequence identities are determined with antibody sequences maximally aligned by the Kabat numbering convention for a variable region or EU numbering for a constant region. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity”, or ADCC, is a mechanism for inducing cell death that depends upon the interaction of antibody-coated target cells (i.e., cells with bound antibody) with immune cells possessing lytic activity (also referred to as effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC (also called CMC) refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

### DETAILED DESCRIPTION

### I. General

The invention provides multispecific agents comprising a first binding arm specifically binding to a first don't eat me receptor and a second binding arm specifically binding to a second don't eat me receptor. Such agents are exemplified by a multispecific agent having first and second arms specifically binding to CD47 and CD24 respectively, and a multispecific agent having first and second binding arms specifically binding to SIRPα and siglec-10 respectively. Targeting both CD47 and CD24 is advantageous in eliminating two don't eat me signals, either of which would reduce elimination of target cells (e.g., cancerous cells) by effector cells. Targeting both CD47 and CD24 from the same agent results in higher discrimination between binding of target cells expressing both CD47 and CD24 and non-target cells expressing only one of these molecules, thus for example reducing binding of a treatment agent to red blood cells, which express CD47 but not CD24. Targeting the counterreceptors of CD47 and CVD24, SIRPα and siglec-10 from a multispecific agent has similar advantages.

### II. Targets

Targets for binding the binding arms of multi-specific agent are don't eat me receptors or their counterreceptors. A don't eat me receptor is a receptor that protects a cell expressing the receptor from the immune system of the organism in which the cell typically resides. Receptors can protect cells from the innate or adaptive immune system or both. CD47 and CD24 protect against the innate immune system.

CD24 (Swiss Prot P25063) is a glycosylphosphatidylinositol (GPI)-anchored cell surface protein having 31 amino acids with 16 potential O- and N-glycosylation sites (in the human) in the mature protein. Side chains include α2, 3 and α2, 6 sialic acid, Lewis X antigen and HNK-1 carbohydrate. Human CD24 is first expressed as an 80 amino acid precursor, from which a signal peptide occupying residues 1-26 and a propeptide corresponding to residues 60-80 are removed from the mature form. Antibodies against CD24 can specifically bind to the protein core or sialic acid side chains or both. It is thought that CD24 interactions with siglec-10 are mediated at least in part by α2, 3 and α2, 6 sialic acids.

CD47 (Swiss Port Q08722) is a membrane bound glycosylated receptor including five transmembrane domains, three cytoplasmic (residues 163-176, 229-235, and 290-323) and three extracellular domains (residues 19-141, 198-207 and 257-268). Residues 1-18 are a signal peptide. The receptor has glycosylation and phosphorylation sites.

SIRPα (Swiss Prot P78324) is a receptor of 496 amino acid, of, which residues 1-20 are a signal peptide, residues 31-373 are an extracellular domains, residues 374-394 are a transmembrane domain and residues 395 to 504 are cytoplasmic. The receptor has glycosylation and phosphorylation sites.

Siglec-10 (Swiss Prot Q96LC7) is a receptor of 697 amino acids of which residues 1-16 are a signal peptide, residues 17-550 are an extracellular domain, residues 551-571 are transmembrane and residues 572-697 are cytoplasmic. The receptor includes disulfide bonds, and glycosylation and phosphorylation sites.

Unless otherwise apparent from the context, reference to a specific target should be understood as referring to human forms, particularly the human form of the provided Swiss Prot accession number. However, non-human forms, such as those of laboratory (e.g., mouse, rat), companion animals or farm animals, can also be used.

Factors relevant to pairing of don't eat me receptors or their counterreceptors for targeting by a multispecific agent include presence of a comparable size sink of off-target molecules (in other words, when the target is cells of a cancer, off-target molecules are those expressed on any of the normal cells of a subject being treating) and suppressing immune responses by the same mechanism, such as via the SHP-1 pathway.

Optionally, multispecific agents incorporate a third binding arm specifically binding to a cancer-associated antigen co-expressed on cancer cells with don't eat me receptors targeted by the other binding arms. The presence of the third arm promotes killing of cancer cells by immune effector cells. Examples of tumor cells antigens include e.g. CD19, CD96, CD20, CD22, CD33, CD38, CD52, CD123, CD44, EGFR, VEGFR, BRCA1 and -2, PSMA, PD-L1, PSA, CEA, HER-2, Mart1/MalanA, Erbb2, IL-17R, PDGFR-α, SLMF7, GD2, CTLA-4, RANKL, and EpCAM.

### III. Exemplary Antibodies or ECDs

Multispecific agents are formed from pairs of heavy and light chain variable regions from component antibodies, and/or ECDs of a don't eat me receptor. The binding arms of a multi-specific agent can have about the same affinity (e.g., within a factor of 2, 3 or 4 or different affinities for their targets (difference greater than 5-fold or 10-fold). For a multi-specific targeted against first and second don't eat me receptors with different ratios of receptors on and off target cells, it is preferred that the binding arm binding to the receptor with the higher ratio of target to off-target molecules have a higher affinity that the binding arm binding to the other receptor to minimize off-target binding of the agent.

Component antibodies can be rodent, chimeric, veneered, humanized, primatized, primate or human among others. The component antibodies can be of the same or different types; for example, one can be humanized and the other human.

The production of other non-human monoclonal antibodies, e.g., murine, guinea pig, primate, rabbit or rat, against an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332.

Antibodies are screened for specific binding to an intended target (e.g., CD47, CD24, SIRPα or siglec-10). Antibodies may be further screened for binding to a specific region of the target, competition with a reference antibody, antagonism of cells bearing the target binding to a ligand or counter receptor (e.g., antagonism of CD47 binding to SIRPα or CD24 binding to siglec-10). Some antibodies antagonize CD47 binding to SIRPα or CD24 binding to siglec-10 with an IC50 of less than 10, 5 or 1 ug/ml. Non-human antibodies can be converted to chimeric, veneered or humanized forms as described above.

Other antibodies have the same heavy and light chain variable regions or same six CDRs as defined by Kabat, or alternative definitions, such as Chothia, composite of Chothia and Kabat, AbM or Contact (see world wide web bioinf.org.uk/abs), or binding to the same epitope or competing for binding with any of these antibodies to their target protein can also be used.

Examples of suitable anti-CD47 antibodies include clones B6H12, 5F9, 8B6, C3, (for example as described in WO 2011/143624) CC9002 (Vonderheide, Nat Med 2015; 21: 1122-3, 2015), SRF23 (Surface Oncology) and ZF1, Zeng et al., Oncotarget. 2016 Dec. 13; 7(50): 83040-83050. Suitable anti-CD47 antibodies include human, humanized or chimeric versions of such antibodies, antibodies having the same heavy and light chain variable regions, or six CDRs of such antibodies, and antibodies binding to the same epitope or competing therewith for binding to CD47. Humanized antibodies (e.g., hu5F9-IgG4-WO2011/143624 or magrolimab) are especially useful for in vivo applications in humans due to their low antigenicity. Some humanized antibodies specifically binds to human CD47 comprising a variable heavy (VH) region containing the VH complementarity regions, CDR1, CDR2 and CDR3, respectively set forth in SEQ ID NO: 20, 21 and 22; and a variable light (VL) region containing the VL complementarity regions, CDR1, CDR2 and CDR3, respectively set forth in SEQ ID NO:23, 24 and 25 of WO2011/143624 (SEQ ID NOS:1-6 herein). Some humanized antibodies include a heavy chain variable region selected from SEQ ID NO: 36, SEQ ID NO: 37 and SEQ ID NO: 38 and a light chain variable region selected from SEQ ID NO: 41, SEQ ID NO: 42 and SEQ ID NO: 43 set forth in WO2011/143624 (SEQ ID NOS. 7-12 herein). Similarly caninized, felinized antibodies and the like are especially useful for applications in dogs, cats, and other species respectively.

Suitable anti-SIRPα antibody specifically binds SIRPα (without activating/stimulating enough of a signaling response to inhibit phagocytosis) and inhibit an interaction between SIRPα and CD47. Suitable anti-SIRPα antibodies include fully human, humanized or chimeric versions of such antibodies. Exemplary antibodies are KWAR23 (Ring et al., Proc. Natl. Acad. Sci. USA. 2017 Dec. 5; 114(49): E10578-E10585. WO/2015/138600, MY-1, Effi-DEM (Zhang et al., Antibody Therapeutics, Volume 1, Issue 2, 21 Sep. 2018, Pages 27-32). Antibodies sharing the same heavy and light chain variable regions or the same six CDRs or competing for binding to SIRPα or binding to the same epitope as any of these antibodies can also be used.

Humanized antibodies are especially useful for in vivo applications in humans due to their low antigenicity. Similarly caninized, felinized, and the like antibodies are especially useful for applications in dogs, cats, and other species respectively.

Soluble CD47 polypeptides that specifically binds SIRPα and reduce the interaction between CD47 on a cancer cell and SIRPα on a phagocytic cell can be used in place of SIRPα antibodies (see, e.g., WO2016179399). Such polypeptides can include the entire ECD or a portion thereof with the above functionality. A suitable soluble CD47 polypeptide specifically binds SIRPα without activating or stimulating signaling through SIRPα because activation of SIRPα would inhibit phagocytosis. Instead, suitable soluble CD47 polypeptides facilitate the phagocytosis of cancer cells. A soluble CD47 polypeptide can be fused to an Fc (e.g., as described in US20100239579).

Likewise soluble SIRPα polypeptides can be used in place of antibodies against CD47. Exemplary agents include ALX148 (Kauder et al., Blood 2017 130:112) and TTI-622 and TTI-661 Trillium). Such agents can include the entire SIRPα ECD or any portion thereof with the above functionality. The SIRPα reagent will usually comprise at least the d1 domain of SIRPα. The soluble SIRPα polypeptide can be fused to an Fc region. Exemplary SIRPα polypeptides termed “high affinity SIRPα reagent”, which includes SIRPα-derived polypeptides and analogs thereof (e.g., CV1-hIgG4, and CV1 monomer are described in WO2013/109752. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, and so forth. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Antibodies against CD24 can bind to an epitope within the protein core or to an epitope within one or more sialic acid side chains or an epitope to which both the protein core and one or more sialic acid side chains contribute. Such antibodies can be generated using an immunogen, which is a purified CD24 protein, or a peptide component thereof, or cells expressing CD24 in which the CD24 is linked to the cell surface via a phosphatidylinositol linker. If cells are used the cells can be cells of a cancer, including cells from a specific subject to be treated for the cancer, or can be a cell line expressing CD24. CD24 antibodies having activity against cancer have been described by WO2009063461 and WO2008002112. Many antibodies against CD24 are commercially available (see world wide web biocompare.com/pfu/110447/soids/3585/Antibodies/CD240. Examples of such antibody specifically binding to human C24 including MAB5248 and AF5247 (R & D Systems), 32D12 (Stem Cell Technologies) and SN3 (BioRad). Antibodies sharing the same heavy and light chain variable regions or the same six CDRs or competing for binding to CD24 or binding to the same epitope as any of these antibodies can also be used.

Soluble forms of CD24, optionally, linked to an Fc fusion protein have been described in several patent publications in the context of treatment of autoimmune disease. These patent publications include WO2001072355, WO2011113047, WO2018217659, WS02018213266 and WO2018105204.

Antibodies against siglec-10 can be generated by immunizing with siglec-10 protein, an extracellular domain thereof or peptides from the extracellular domain or cells expressing siglec-10. Antibodies can be screened for specific binding to siglec-10 and optionally lack of specific binding to other siglecs (e.g., siglecs 1-9 and 11-16). Antibodies can then be screened for inhibiting of binding to CD24 or sialic acids modified CD24. Examples of antibodies specifically binding to siglec-10 and not other siglecs and inhibiting binding of siglec-10 to sialic acids are described by WO2017085166. Antibodies against human siglec-10 are also commercially available (see world wide web biocompare.com/pfu/110447/soids/331695/Antibodies/SIGLEC10). Examples of such antibodies include AF2130 from R&D Systems, 5G6 from Bio-Rad, and NBP1-59247 from Novus Biologicals. Antibodies sharing the same heavy and light chain variable regions or the same six CDRs or competing for binding to siglec-10CD24 or binding to the same epitope as any of these antibodies can also be used.

### IV. Formats for Multispecific Agents

Over 100 formats have been described for bispecific or multispecific agents (e.g., Kontermann et al., Drug Discovery Today 20, 838-847 (2015); Sedykh et al., Drug Des. Devel. Ther. 2, 195-209 (2018)). Such formats including at least one binding site for each of two targets. Some formats includes two or more binding sites for each target. Some formats include one or more binding sites for each of at least three targets.

Some formats have a similar tetrameric structure to a normal antibody with two different binding regions, one for each of two targets. Each binding region is formed from paired heavy and light chain variable regions, which are linked to heavy and light chain constant regions respectively. Such bispecific antibodies differ from a normal antibody in that the two binding sites and pairs of heavy and light chains forming them are different. Thus, such antibodies require association of two different pairs of heavy and light chains.

The “knobs-into-holes” approach has been adopted to reduce formation of homodimers and mispairing of heavy chains by substituting a large amino acid for a small one in the CH3 domain (the “knob”) of one antibody and vice versa (the “hole”) of the other antibody (Ridgway et al., Protein Eng. 9:617-21, 1996; Atwell et al., J. Mol. Biol. 270:26-35, 1997; and U.S. Pat. No. 7,695,936). Light chain mispairing in such formats can be reduced by a number of strategies. One strategy is to use a common light chain variable region for two different heavy chain variable regions. But this is applicable only to some antibodies. Another approach has been to express the knob- and the hole-containing half-molecules separately in different bacteria. Another approach termed CrossMab swaps the CH1 domain of one of the heavy chain the constant CL domain of the corresponding light chain to induce the right pairing of the light chains. Schaefer et al., Proc. Natl. Acad. Sci. USA 108:11187-92, 2011; WO 2009/080251; WO 2009/080252; WO 2009/080253). Another approach has been to introduce additional mutations into VH-VL and CH1-CL interfaces. Lewis et al., Nat. Biotechnol. 32, 191-198 (2014). These mutations encourage a heavy chain to preferentially pair with a light chain. Another approach has been to introduce mutations promoting protein A binding into one of the Fc regions and select heterodimeric pairing having intermediate protein A binding from homodimers having higher or lower protein A binding by affinity chromatography (Tusdian et al, MAbs. 8(4):828-38 (2016)).

Other bispecific antibodies avoid the problem of mispairing by combining multiple binding specificities in the same heavy light chain pair. One approach for doing this, termed dual variable domains is to link two different heavy chain variable regions in tandem to a heavy chain constant region and two different light chain variable regions in tandem to a light chain constant region. Correia et al., MAbs. 2013 May 1; 5(3): 364-372. Such an antibody can assemble as tetramer by association of two identical paired heavy and light chains. The assembled antibody include two different binding sites for each target.

Another approach is to incorporate a second binding specificity by linking an scFv to the C-terminus of a heavy chain constant region. Such a bispecific includes a first binding site formed by heavy and light chain variable regions attached to the N-termini of heavy and light chain constant regions as in a standard antibody. The C-terminus of the heavy chain is attached to a scFv providing the second binding site. The scFv is usually attached via a linker and a further linker connects the heavy and light chain variable regions in the scFv. The scFv can be attached either through its light chain variable region or heavy chain variable region end via the linker to the Fc region. When assembled by complexing of two identical paired heavy and light chains, such a bispecific includes two binding sites for each of two different specificities. The antibodies for the respective targets can be attached in either orientation. The arm to be attached to the N-termini of the heavy and light chain constant regions is provided as separate heavy and light chain variable regions, and that to be attached to the C-terminus is provided as an scFv fragment.

Another format links an scFv specifically binding to first target to a heavy chain constant region and an scFv specifically binding to another target to a light chain constant region. Such an antibody assembles into a tetramer including two copies of each binding site (Bs(scFv)4-IgG). (Zuo et al., Protein Eng 13: 361-367, 2000).

Other formats link scFv binding regions on a single chain without a constant region. For example, the BiTe format links two scFv fragments through a linker (see, e.g., Ross et al., PLoS ONE 12(8): e0183390, 2017). Such formats lack effector functions and tend to have a short half-life but may have advantages of accessibility and ease of manufacture due to their small size. Another format links two or more different scFv's to an Fc domain, usually to its N-terminus.

In any of the above formats, an antibody binding arm including a heavy and light chain variable region can be replaced by an ECD of a don't eat me receptor or its counterreceptor. Usually, the ECD is fused to an Fc domain in such a format.

Many of the formats can be extended to multispecifics having three or more binding arms (see, e.g., Runcie et al., Mol. Med 24, 50 (2018), Steinhardt et al., Nature Communications 9, 877 (2018), Hu et al., Cancer Res, 75, 159-170 (2015)). For example, bispecific formats having the same tetrameric structure as a normal antibody can be extended to trimeric or higher, by including an scFv on either of the heavy or light chains or on the C-terminus of the Fc fragment. Alternatively, separate heavy and light chain variable regions encoding a third binding arm can be fused to heavy and light chain variable region of the tetrameric antibody structure. The BiTe format can likewise be extended by fusing three more scFv's in tandem.

The ECD of a counterreceptor behaves similarly to antibodies to the receptor, and the ECD of the receptor behaves similarly to antibodies to the counterreceptor. The ECD should include sufficient sequence from the extracellular portion of a receptor so as to retain the ability to bind the ligand or counterreceptor of the receptor.

Many of the above formats include linker peptides between heavy and light variable regions or between variable regions and a constant region. Linkers are short peptide conferring flexibility often predominantly occupied by gly, ala and/or ser. Some exemplary linkers are Gly-Gly-Ala-Ala, Gly-Gly-Gly-Gly-Ser, Leu-Ala-Ala-Ala-Ala and multimers thereof.

### V. Selection of Constant Region

Many of the formats for a multispecific agents include at least a portion of a human constant region or Fc portion thereof. The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4. For the present multispecific agent, IgG4, IgG2 or an attenuated IgG1 with reduced effector function are generally preferred because Here although ADCC, ADCP and CDC may be useful in providing an additional mechanism of action against cancer cells bound by one arm of a multispecific agents, it also increases toxicity to off-target cells. Thus, human IgG4, IgG2 or mutated IgG1 with reduced effector function is preferred in some multispecific agents.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC or remove a glycosylation site (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). For example, there are many known mutations in IgG Fc that increase FcRn binding. Exemplary substitutions include a Gln at position 250 and/or a Leu at position 428, Ser or Asn at position 434, Tyr at position 252, Thr at position 254, and Glu at position 256, and Ala at position 434 (EU numbering). Increased FcRn binding is advantageous in making the hybrid proteins of the present invention compete more strongly with endogenous IgG for binding to FcRn. Also numerous mutations are known for reducing any of ADCC, ADCP or CMC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). For example, substitution any of positions 234, 235, 236 and/or 237 reduce affinity for Fcγ receptors, particularly FcγRI receptor (see, e.g., U.S. Pat. No. 6,624,821). Optionally, positions 234, 236 and/or 237 in human IgG2 are substituted with alanine and position 235 with glutamine or glutamic acid. (See, e.g., U.S. Pat. No. 5,624,821.) Other substitutions reducing effector function include Ala at position 268, Gly or Ala at position 297, Leu at position 309, Ala at position 322, Gly at position 327, Ser at position 330, Ser at position 331, Ser at position 238, Ala at position 268, Leu at position 309.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes.

### VI. Expression of Recombinant Multispecific Agents

Multispecific agents are typically produced by recombinant expression. Depending on the format, expression may be required for one, two or more antibody chains and or ECD domains. If multiple chains are expressed, they can be expressed from the same or different vectors. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector has been incorporated into the appropriate host, the host is maintained under conditions suitable for high level expression of the nucleotide sequences and the collection and purification of the multispecific agents.

These expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, N Y, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NS0. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, sequences encoding multispecific agents can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains, or ECDs operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells, and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 pg or 100 pg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, multispecific agents can be purified according to standard procedures of the art, including protein A capture, HPLC purification, column chromatography, gel electrophoresis and the like (see generally, Scopes, Protein Purification (Springer-Verlag, NY, 1982)).

Methodology for commercial production of antibodies or Fc fusion proteins can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. Nos. 5,786,464; 6,114,148; 6,063,598; 7,569,339; WO2004/050884; WO2008/012142; WO2008/012142; WO2005/019442; WO2008/107388; WO2009/027471; and U.S. Pat. No. 5,888,809).

### VII. Nucleic Acids

The invention further provides nucleic acids encoding any of the antibody or ECD chains described above. Optionally, such nucleic acids further encode a signal peptide and can be expressed with the signal peptide linked to the constant region Coding sequences of nucleic acids can be operably linked with regulatory sequences to ensure expression of the coding sequences, such as a promoter, enhancer, ribosome binding site, transcription termination signal, and the like. The nucleic acids encoding heavy and light chains or ECDs can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

### VIII. Methods of Treatment and Pharmaceutical Compositions

The multispecific agents of the invention can be used for treating cancers. Some cancers have cells concurrently expressing each of the targets of a multispecific agent, such as CD24 and CD47. However, some multispecific agents against e.g., SIRPα and siglec-10, can also be used to promote action of effector cells expressing SIRPα and siglec-10 against a cancerous cell. The multispecific agents can be used to treat solid tumors, and hematological malignancies. Hematological malignancies include leukemia (e.g., acute or chronic myeloid or myelogenous leukemia, acute lymphoblastic or lymphocytic leukemia), lymphoma (Hodgkin's or Non-Hodgkin's), or multiple myeloma. Solid tumors carcinomas, sarcoma, adenocarcinomas. Solid tumors can occur be of the skin (e.g., melanoma), ovarian, endometrial, kidney, liver, pancreas, bladder, breast, ovarian, prostate, rectum, colon, gastric, intestinal, pancreatic, lung, thymus, thyroid, kidney, brain, bones. Examples of cancers showing higher expression of CD24 than matched normal tissue include cervical squamous cell carcinoma, cholangiocarcinoma, kidney renal papillary cell carcinoma, liver hepatocellular carcinoma, lung adenocarcinoma, lung squamous cell carcinoma, pheochromocytoma and paraganglioma, and uterine corpus endometrial carcinoma. Several lymphomas also show higher expression of CD24 than tissue matched noncancerous cells including diffuse large B-cell lymphoma (DLBCL) in which siglec-1 is also upregulated. Examples of cancers expressing CD47 at higher levels than normal tissues include leukemia (e.g., acute myeloid leukemia (AML) and acute lymphoblastic leukemia (ALL), and solid tumor cancers, e.g., breast, bladder, colon, ovarian, glioblastoma, leiomyosarcoma, and head & neck squamous cell carcinomas.

Multispecific agents are administered in an effective regime meaning a dosage, route of administration and frequency of administration that delays the onset, reduces the severity, inhibits further deterioration, and/or ameliorates at least one sign or symptom of a condition. If a subject is already suffering from a disorder, the regime can be referred to as a therapeutically effective regime. If the subject is at elevated risk of the condition relative to the general population but is not yet experiencing symptoms, the regime can be referred to as a prophylactically effective regime. In some instances, therapeutic or prophylactic efficacy can be observed in an individual subject relative to historical controls or past experience in the same subject. In other instances, therapeutic or prophylactic efficacy can be demonstrated in a preclinical or clinical trial in a population of treated subjects relative to a control population of untreated subjects.

Preferably a multispecific agent exhibits at least additive and more preferably synergistic activity against a cancer compared with its component binding arms individually. Synergy is preferably assessed quantitatively such as discussed by Tallarida, Genes Cancer. 2011 November; 2(11): 1003-1008. Preferably a multispecific agent also exhibits increased activity compared with a mixture of its component binding arms, each at equimolar concentration with the multispecific agent. Such activity can be measured, for example, as cytotoxicity against cancer cells or infected cells expressing don't eat me receptors specifically bound by arms of the multispecific agent in the presence of immune cell expressing counterreceptors of the multispecific agent.

Exemplary dosages for a multispecific agent are 0.01-20, or 0.5-5, or 0.01-1, or 0.01-0.5 or 0.05-0.5 mg/kg body weight (e.g., 0.1, 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration into the systemic circulation by intravenous or subcutaneous administration is preferred. Intravenous administration can be, for example, by infusion over a period such as 30-90 min.

The frequency of administration depends on the half-life of the multispecific agent in the circulation, the condition of the subject and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of chronic disorders between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, a multispecific agent can be administered at regular intervals, e.g., weekly, fortnightly, monthly, quarterly, every six months for at least 1, 5 or 10 years, or the life of the subject.

Pharmaceutical compositions are preferably suitable for parenteral administration to a human (e.g., according to the standard of the FDA). Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more pharmaceutically acceptable carriers, diluents, excipients or auxiliaries. Pharmaceutically acceptable means suitable for human administration, e.g., approved or approvable by the FDA. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

Treatment with the multispecific agents of the invention can be combined with other treatments effective against the disorder being treated. When used in treating cancer, the multispecific agents can be combined with chemotherapy, radiation, stem cell treatment, surgery or treatment with other biologics such as Herceptin™ (trastuzumab) against the HER2 antigen, Avastin™ (bevacizumab) against VEGF, or antibodies to the EGF receptor, such as (Erbitux™, cetuximab), and Vectibix™ (panitumumab). Chemotherapy agents include chlorambucil, cyclophosphamide or melphalan, carboplatinum, daunorubicin, doxorubicin, idarubicin, and mitoxantrone, methotrexate, fludarabine, and cytarabine, etoposide or topotecan, vincristine and vinblastine.

### IX. Other Methods

The multispecific agents of the invention also find use in diagnostic, prognostic and laboratory methods. They may be used to measure the level of an antigen expressed by a cancer or in the circulation of a patient with a cancer, to determine if the level is measurable or even elevated, and therefore to follow and guide treatment of the cancer, because cancers associated with measurable or elevated levels of an antigen are most susceptible to treatment with a multispecific agent comprising an arm binding to the cancer. The multispecific agents can be used for an ELISA assay, radioimmunoassay or immunohistochemistry among others. The multispecific agents can be can be labeled with fluorescent molecules, spin-labeled molecules, enzymes or radioisotopes, and may be provided in the form of kit with all the necessary reagents to perform the assay.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1: Expression of CD24 and Siglec-10

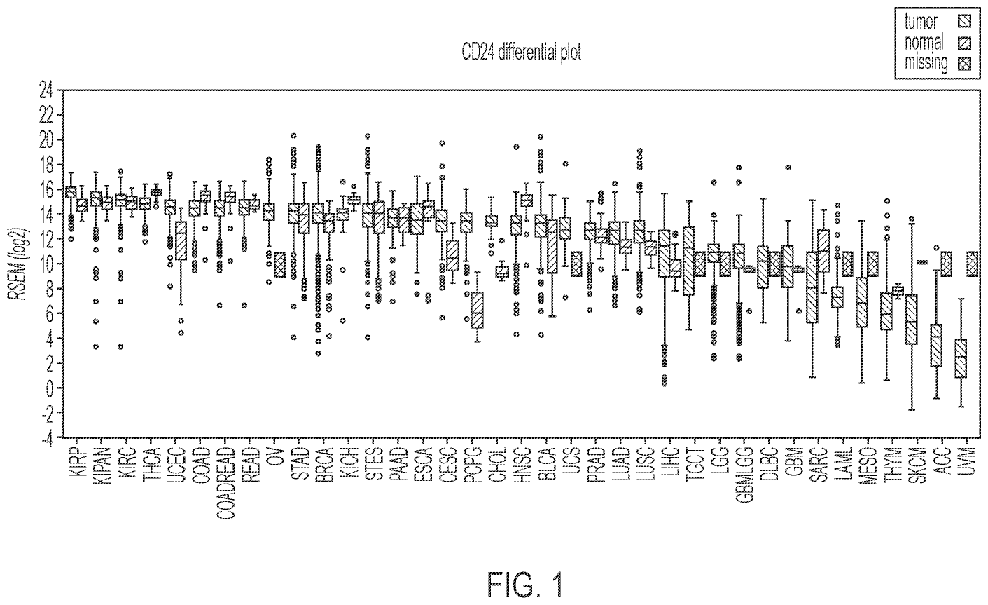

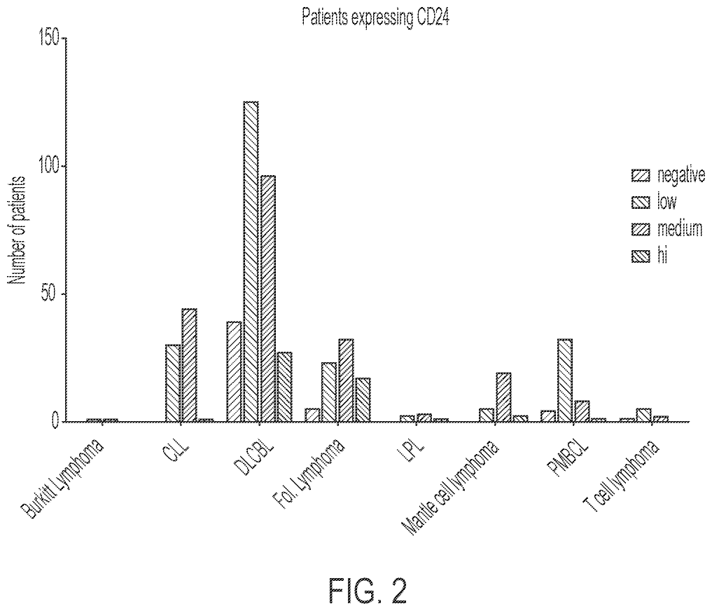

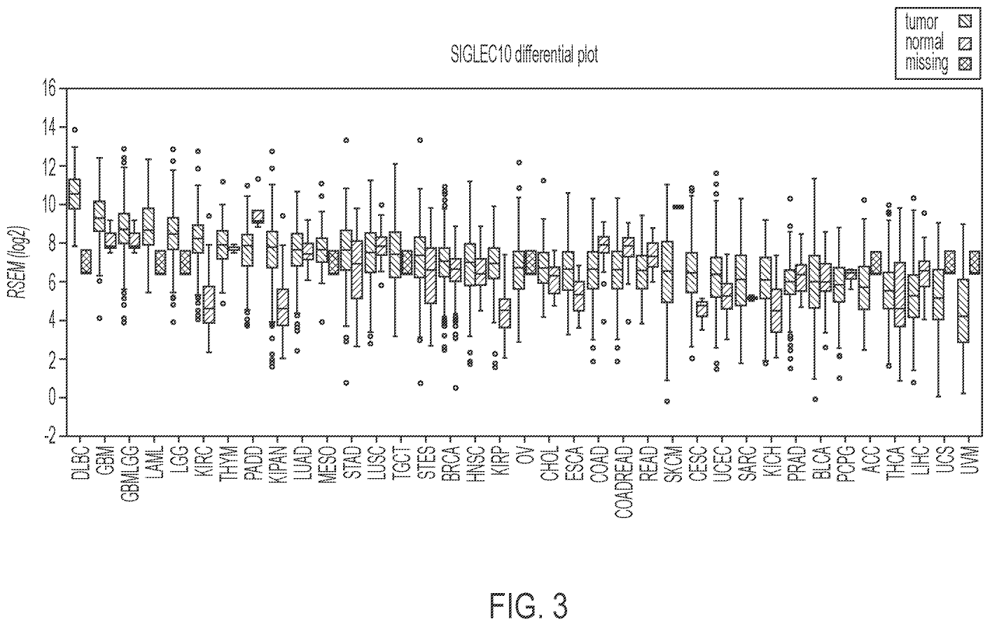

This examples compares CD24 expression in cancers of various tissues compared with matched noncancerous tissue. The highest differential expression of CD24 in cancers over normal tissue was seen in cervical squamous cell carcinoma, cholangiocarcinoma, kidney renal papillary cell carcinoma, liver hepatocellular carcinoma, lung adenocarcinoma, lung squamous cell carcinoma, pheochromocytoma and paraganglioma, uterine corpus endometrial carcinoma (FIG. 1). CD24 is also expressed at varying levels in several lymphomas, particularly CLL, DLCSBL, follicular lymphoma, mantle cell lymphoma, and PMBCL (FIG. 2). Expression of siglec-10 occurs mainly in hematopoietic cells and lymphatic tissue of normal subjects. Siglec-10 is differentially expressed in several cancers particularly DLBCL (FIG. 3).

### Example 2

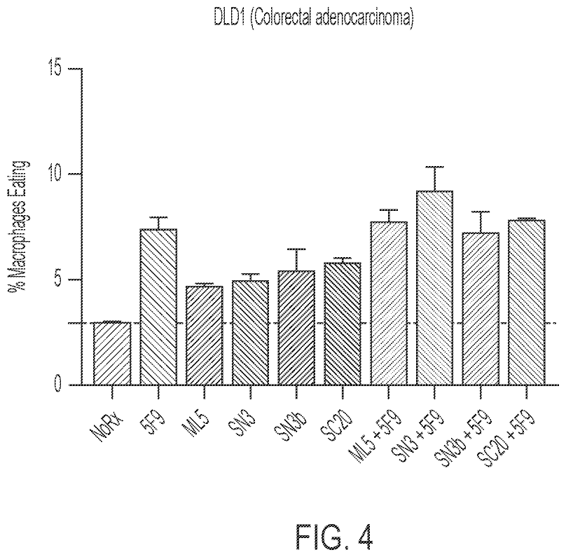

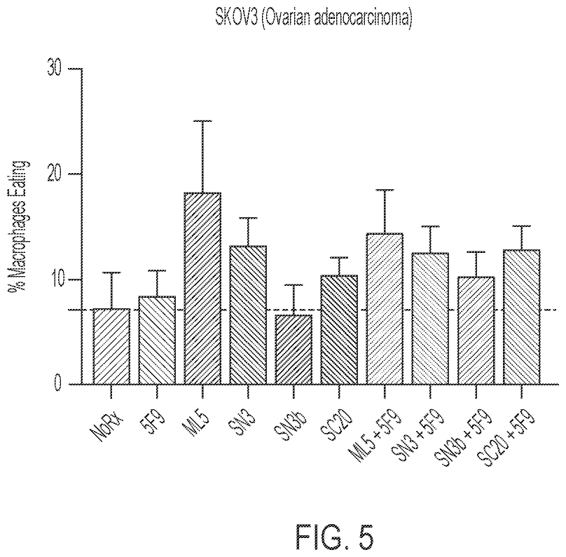

FIGS. 4 and 5 show macrophage eating of cancerous cells induced by various antibodies. 5F9 is a monoclonal antibody against CD47. ML5 (Novus), SN3 and SN3B (Thermo Fisher) and SC20 (Uchid, PNAS 97, 14720 (2000) are antibodies against CD24.

What is claimed is:

1. A multispecific agent comprising a first binding arm that specifically binds CD47 and a second binding arm that specifically binds to CD24.

2. The multispecific agent of claim 1, wherein the first binding arm antagonizes CD47 binding to SIRPα and the second binding arm antagonizes CD24 binding to siglec-10.

3. The multispecific agent of any preceding claim, wherein the first binding arm is an antibody VH-VL pair or a SIRPα extracellular domain, and second binding arm is an antibody VH-VL pair or a siglec-10 extracellular domain.

4. The multispecific agent of any preceding claim having a single first binding arm and a single second binding arm.

5. The multispecific agent of any one of claims 1-3 having two copies of a first binding arm and two copies of a second binding arm.

6. The multispecific agent of any preceding claim, further comprising a third binding arm specifically binding to a cancer antigen

7. The multispecific agent of claim 6, wherein the cancer antigen is CD20.

8. The multispecific agent of any preceding claim, wherein the first and second binding arms have affinities for CD47 and CD24 within a factor of four for one another.

9. The multispecific agent of any one of claims 1-7, wherein the second binding arm has a higher affinity for CD24 by at least a factor of five than the first binding arm has for CD47.

10. The multispecific agent of any preceding claim, further comprising an Fc domain.

11. The multispecific agent of claim 10, wherein the Fc domain is of human IgG4 isotype.

12. The multispecific agent of claim 10, wherein the Fc domain of human IgG1 or IgG4 isotype.

13. The multispecific agent of claim 10, which is of human IgG1 isotype mutated to reduce effector functions.

14. A method of treating a patient having a cancer, comprising administering a multispecific agent of any preceding claim to the patient.

15. The method of claim 14, wherein the cancer expresses CD24 and CD47.

16. The method of claim 13 or 15, wherein the multispecific agent further comprises a third binding arm specifically binding to a cancer antigen, wherein the cancer expresses the cancer specific antigen.

17. The method of any one of claims 14-16, wherein the cancer is adenocarcinoma.

18. The method of any one of claims 14-16, wherein the cancer is a lymphoma.

19. The method of any one of claims 14-18, further comprising detecting expression of CD24 and CD47 on cells of the cancer.

20. A multispecific agent comprising a first binding arm that specifically binds to SIRPα and a second binding arm that specifically binds to siglec-10.

21. The multispecific agent of claim 20, wherein the first binding arm antagonizes CD47 binding to SIRPα and the second binding arm antagonizes CD24 binding to siglec-10.

22. The multispecific agent of claim 20 or 21, wherein the first binding arm is an antibody VH-VL pair or a SIRPα extracellular domain, and second binding arm is an antibody VH-VL pair or a siglec-10 binding domain.

23. The multispecific agent of any one of claims 20-22 having a single first binding arm and a single second binding arm.

24. The multispecific agent of any one of claims 20-22 having two copies of a first binding arm and two copies of a second binding arm

25. The multispecific agent of any one of claims 20-24, wherein the first and second binding arms have affinities for SIRPα and siglec-10 within a factor of four for one another.

26. The multispecific agent of any one of claims 20-24, wherein the second binding arm has at least five fold higher affinity for siglec-10 than the first binding arm has for SIRPα.

27. The multispecific agent of any one of claims 20-27, further comprising an Fc domain.

28. The multispecific agent of claim 27, wherein the Fc domain is of human IgG4 isotype.

29. The multispecific agent of claim 27, wherein the Fc domain of human IgG1 or IgG4 isotype.

30. The multispecific agent of claim 27, wherein the Fc domain is of human IgG1 isotype mutated to reduce effector functions.

31. A method of treating a patient having a cancer, comprising administering a multispecific agent of any one of claims 20-30 to the patient.

32. The method of claim 31, wherein the multispecific agent further comprises a third binding arm specifically binding to a cancer antigen, wherein the cancer expresses the cancer specific antigen.


SEQUENCE LISTING (12 sequences)

SEQ ID NO: 1 - protein, 5 aa, Artificial Sequence
  5F9 heavy chain CDR1
NYNMH

SEQ ID NO: 2 - protein, 17 aa, Artificial Sequence
  5F9 heavy chain CDR2
TIYPGNDDTSYNQKFKD

SEQ ID NO: 3 - protein, 8 aa, Artificial Sequence
  5F9 heavy chain CDR3
GGYRAMDY

SEQ ID NO: 4 - protein, 16 aa, Artificial Sequence
  5F9 light chain CDR1
RSSQSIVYSNGNTYLG

SEQ ID NO: 5 - protein, 7 aa, Artificial Sequence
  5F9 light chain CDR2
KVSNRFS

SEQ ID NO: 6 - protein, 9 aa, Artificial Sequence
  5F9 light chain CDR3
FQGSHVPYT

SEQ ID NO: 7 - protein, 117 aa, Artificial Sequence
  Humanized antibody hu5F9-vh1
QVQLVQSGAEVKKPGASVKVSCKASGYTFTNYNMHWVRQAPGQGLEWIGTIYPGNDDTSYNQKFKDKATLTADKSTSTAYMELSSLRSEDTAVYYCARGGYRAMDYWGQGTLVTVSS

SEQ ID NO: 8 - protein, 117 aa, Artificial Sequence
  Humanized antibody hu5F9-vh2
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 9 - protein, 117 aa, Artificial Sequence
  Humanized antibody hu5F9-vh3
QVQLVQSGAEVKKPGASVKVSCKASGYTFTNYNMHWVRQAPGQRLEWIGTIYPGNDDTSYNQKFKDRATLTADKSASTAYMELSSLRSEDTAVYYCARGGYRAMDYWGQGTLVTVSS

SEQ ID NO: 10 - protein, 112 aa, Artificial Sequence
  Humanized antibody hu5F9-vl1
DVVMTQSPLSLPVTPGEPASISCRSSQSIVYSNGNTYLGWYLQKPGQSPKLLIYKVSNRFSGVPDRFSGSGSGTDFTLKISRVEAEDVGVYHCFQGSHVPYTFGGGTKVEIK

SEQ ID NO: 11 - protein, 112 aa, Artificial Sequence
  Humanized antibody hu5F9-vl2
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 12 - protein, 112 aa, Artificial Sequence
  Humanized antibody hu5F9-vl3
DVVMTQSPLSLPVTPGEPASISCRSSQSIVYSNGNTYLGWYLQKPGQSPQLLIYKVSNRFSGVPDRFSGSGSGTDFTLKISRVEAEDVGVYHCFQGSHVPYTFGQGTKLEIK